(19) United States
(12) Patent Application Publication
(10) Pub. No.: US 2016/0354222 A1
(43) Pub. Date: Dec. 8, 2016

(54) FLEXIBLE PNEUMATIC SPLINT

(71) Applicant: Umm Al-Qura University, Makkah (SA)

(72) Inventor: Badr ALSOLAMI, Makkah (SA)

(73) Assignee: Umm Al-Qura University, Makkah (SA)

(21) Appl. No.: 14/731,992

(22) Filed: Jun. 5, 2015

(51) Int. Cl. A61F 5/01

(52) U.S. Cl. CPC A61F 5/012

(57) ABSTRACT

A splint for immobilization of a limb of a patient. The splint includes an outer immobilization construct extending in a longitudinal direction of the limb, an inner immobilization construct in contact with the limb, a splint binder binding the outer immobilization construct in place around the second wrapping construct, and an expandable air cuff disposed between the outer immobilization construct and the second immobilization construct, which upon inflation exerts pressure on the second immobilization construct to conform the inner immobilization construct to the limb. With the splint, a method for immobilizing the limb includes placing a conformable restraint over the limb, securing an inflatable air cuff over the conformable restraint, and pressurizing the inflatable air cuff to thereby exert a controlled pressure on the limb.

### BACKGROUND OF THE INVENTION

Field of the Invention

The present invention is related to orthopedic splints for securing a limb in place.

Description of the Related Art

Many configurations of limb splints are known.

Splints for use in emergency and post-operative situations are also known, for example, from Scheinberg, U.S. Pat. Nos. 4,676,233 and 3,943,923, both of which teach the use of a soft metal sheet formed into a configuration that provides needed support for an injured limb. Bentele, U.S. Pat. No. 4,161,175, describes surgical splints incorporating formed sheet metal or molded plastic bases. Ender, U.S. Pat. No. 4,549,537, describes another splint based on the use of sheet aluminum material. Bolla, et al., U.S. Pat. No. 6,039,706, describes a splint using a sheet of corrugated metal as its principal supporting structure. The contents of each of these patents and U.S. Pat. No. 7,329,229 (as well as the patent documents below) are incorporated herein in their entirety by reference.

Health care professionals have long used splints and casts fashioned out of plaster, fiberglass, preformed metal, or molded thermoplastic materials. These splints are designed to rigidly prevent motion and once formed into position cannot be reformed, for example to accommodate swelling, without considerable difficulty. A thermosetting plastic or fiberglass-reinforced resin splint once cured cannot be reformed. If swelling is excessive, a new fiberglass or plaster splint must be applied or an instrument resembling large pliers (often referred to as a cast bender) is used to break the plaster or fiberglass material in order to relieve the pressure caused by the swelling.

Where a person received an injury to an ankle or forearm that needs to be immobilized, previously known conformable splint devices for use as described in Scheinberg et al. U.S. Pat. No. 3,943,923 and Scheinberg U.S. Pat. No. 4,676,233, have been widely used. The lack of precise conformation of such splints to the limb allows unwanted rotation and lateral movement, which, in turn, could affect injuries occurring anywhere along the length of the encompassed limb.

U.S. Pat. No. 7,329,229 describes ways to make a conformal splint member.

### SUMMARY OF THE INVENTION

In one embodiment of the present invention, there is provided a splint for immobilization of a limb of a patient. The splint includes an outer immobilization construct extending in a longitudinal direction of the limb, an inner immobilization construct in contact with the limb, a splint binder belt binding the outer immobilization construct in place around the second wrapping construct, and an expandable air cuff disposed between the outer immobilization construct and the second immobilization construct, which upon inflation exerts pressure on the second immobilization construct to conform the inner immobilization construct to the limb.

In one embodiment of the present invention, there is provided a method for immobilizing a limb of a patient. The method places a conformable restraint over the limb, secures an inflatable air cuff over the conformable restraint, and pressurizes the inflatable air cuff to thereby exert a controlled pressure on the limb for immobilization of the limb.

In one embodiment of the present invention, there is provided a medical kit including the above-noted splint and instructions detailing the above-noted method for immobilization.

It is to be understood that both the foregoing general description of the invention and the following detailed description are exemplary, but are not restrictive of the invention.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic depicting an inflatable exoskeleton splint according to one embodiment of the invention;

FIG. 2 is a schematic depicting an inflatable exoskeleton splint according to one embodiment of the invention in which a controller regulates pressure in the splint by control of a pump and a release valve;

FIG. 3 is a schematic depicting an inflatable exoskeleton splint according to one embodiment of the invention in which a controller is in communication with an automated blood pressure measurement device 32;

FIG. 4A is a schematic of a medical kit of the invention;

FIG. 4B is a flowchart depicting a method for immobilization of a limb of a patient; and

FIG. 5 is a schematic depicting a computer system for implementing various embodiments of the invention.

### DETAILED DESCRIPTION OF THE INVENTION

This invention provides a novel type of inflatable exoskeleton limb splint (e.g., a support or cast) for securing a limb in place. In the present context, the term “splint” or “splint structure” is intended to mean a rigid assembly for application to a body part, most commonly a limb, so as to support and maintain parts of the limb in a constant mutual disposition, thereby facilitating repair of a fracture or other tissue damage.

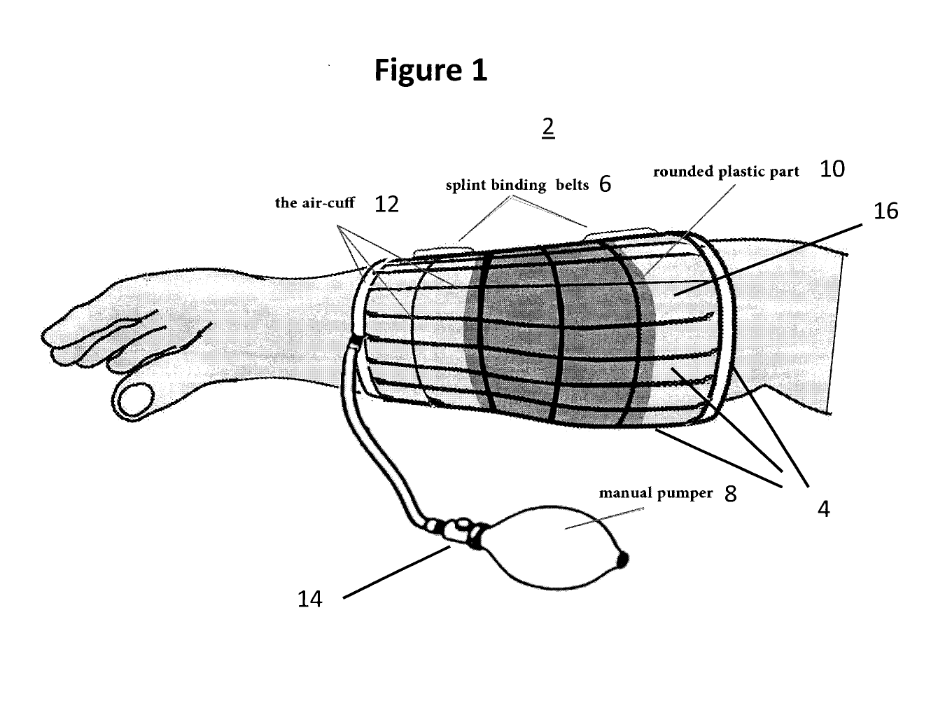

FIG. 1 is a schematic of the exoskeleton splint 2 according to one embodiment of the invention. As shown in FIG. 1, the exoskeleton splint 2 comprises a splint structure 4 for holding the splint in place, one or more splint binding belts 6 (i.e., a splint binder or other fastening device), air pump 8, and an immobilization part 10 (depicted here a “rounded plastic part”) conformable to the limb. The splint 2 can be considered to include at least two wrapping constructs. As viewed from the outside to the inside, the outer immobilization construct (i.e., splint structure 4) extends in a longitudinal direction of the limb and may include an inflatable layer or an air cuff 12. The inner immobilization construct (i.e., immobilization part 10) physically contacts the limb. It may also include the inflatable layer or air cuff 12 or another inflatable layer or air cuff. Regardless of the exact placement of the inflatable layer or the air cuff 12, each of these items is disposed between the outer and inner immobilization constructs, and (by inflation) forces the inner immobilization construct to conform to the limb. By applying pressure to the inflatable layer or the air cuff 12, splint 2 can apply a controlled force on the limb to immobile the limb during healing of a broken bone, bone fracture, or limb injury (e.g., muscle or ligament injury).

The exoskeleton splint shown in FIG. 1 has a construction which the flexible immobilization part 10 is flexible along a direction which permits the splint to conform to the limb upon inflation. Upon inflation, the pressures in the air cuffs (coupled with the tension maintained by the splint binding belts) serves to make the splint resistant to deformation in a direction perpendicular to the limb.

In one embodiment of this invention, multiple (or progressive) pressures can be applied to allow the limb to be secured. If a force exerted greater than the broken bone can resist is applied, the exoskeleton splint can be used to assist in the setting of minor breaks through a controlled shift of the broken bone.

In one embodiment of the invention, air is inflated into a designed inflatable network associated with the outer immobilization construct (as in an integral part of the outer immobilization construct). In one embodiment of the invention, air is inflated into a designed inflatable network associated with the inner immobilization construct (as in an integral part of the inner immobilization construct). In one embodiment of the invention, air is inflated into a designed inflatable network disposed between the outer and inner immobilization parts and not integral to either. In one embodiment of the invention, pump 8 is a manual pump similar to those used in a clinical mercury manometer (e.g., a blood pressure pack). A gauge/release valve 14 is available for the user. In one embodiment of the invention, pump 8 can be an automatic pump such those used to inflate and maintain pressure in air beds or automatic blood pressure measurement equipment.

In one embodiment of the invention, splint structure 4 is made of lightweight materials (as opposed to plaster materials). In one embodiment of the invention, structure 4 is made of made of odor-free fabric materials such as those used in sports wraps including elements such as for example copper or titanium.

In one embodiment of the invention, splint structure 4 can be considered a flexible splint structure made of a framed structure with number of openings 16 permitting pneumatic parts such as the air cuffs 12 to be expanded by air inflation. Once inflated, as noted above, splint 2 becomes a rigid structure resistant to deformation or bending. In one embodiment of the invention, splint 2 can include a pressure regulation and/or release device to release air if the pressure of air within the air-cuff, inside the framed structure, exceeds a predetermined threshold.

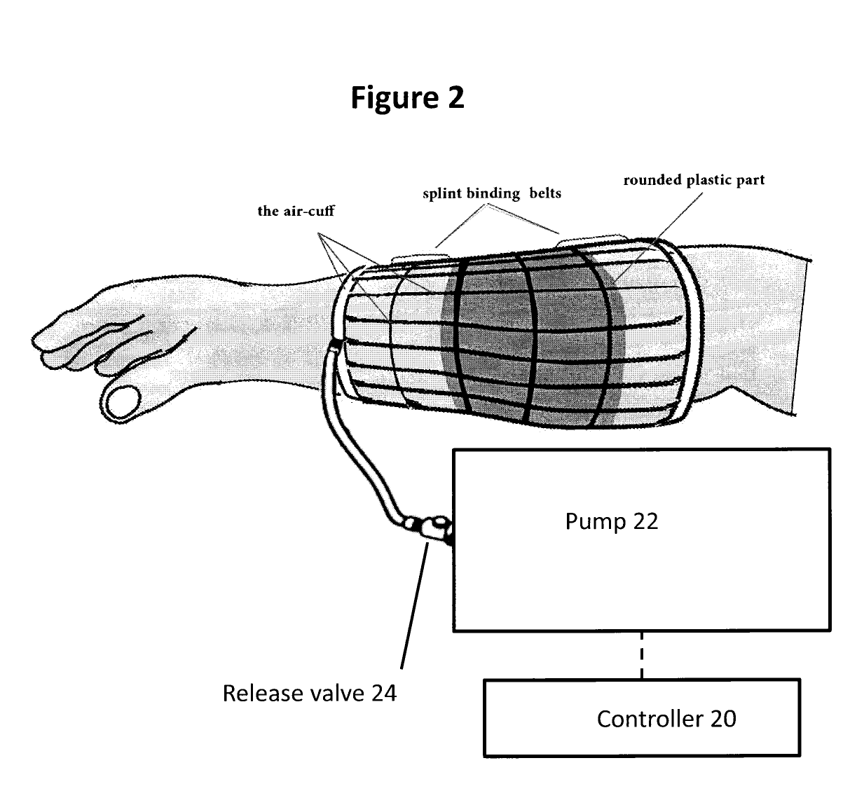

In one embodiment of the invention, as shown in FIG. 2, a controller 20 regulates the pressure by control of a pump 22 and a release valve 24. In one embodiment, this invention can use (especially for field use) a can of compressed air that provides air to flow the designed network of cuff, inside the expanded material which is used in the splint 2.

In use, flexible immobilization part 10 (for example a rounded plastic or metal part) is placed over a focus area of the broken bone or in general around the fractured area or limb. Then, splint structure 4 is wrapped around the flexible immobilization part 10 (and thus around the broken or fractured limb). Splint binding belts 6 bind the flexible immobilization part 10 (i.e., the inner immobilization construct) and the splint structure 4 (i.e., the outer immobilization construct) together. After that, a pump (e.g., manual pump 8 or an automatic pump 22) inflates the inflatable layer or air cuff 12 of the splint structure 4.

In one embodiment of the invention, the pneumatic force not only creates force over the broken or fractured bone, particularly, over the focused area (by exerting a force on flexible immobilization part 10) but also stabilizes the area around the focused area. By this technique of the invention, the splint force is particularly focused on the fractured area of the bone to provide more support.

As can be understood from the above-description, in one embodiment of the invention, splint 2 is adjustable in size to fit an individual properly. A variety of sizes for the splint structures 4 and the flexible parts 10 can be used to further accommodate fitting. In particular, the flexible immobilization part 10 can be made of limb splint materials such as those described in the '229 patent (noted above).

For example, as manufactured, immobilization part 10 can have a generally planar configuration, as used for shipping or storage of this part. Typically, the length of flexible immobilization part 10 is greater than its width. For example, the length may be thirty and forty cm, and the width may be in the range of ten to twelve cm. The flexible immobilization part 10 can include a skin contact made of a material intended to be placed in contact with a patient's skin or wound dressing.

Immobilization part 10 can be made of a metal (having a core of a malleable metal) in the form of a flat sheet whose dimensions are slightly smaller than the length and the width described above. The metal sheet may be folded to provide greater stiffness to the flexible part. Alternatively, immobilization part 10 can be a flexible plastic part, or a plastic part with a core of malleable metal.

In one embodiment of the invention, the core of the immobilization part 10 would include a thicker portion having a length preferably in the range of about 7.5 to 15 cm, for a splint whose length is in the range of about 22 to 37 cm. The greater thickness may be provided by way of folding the aluminum sheet material back upon itself, if the immobilization part is primarily metal. Indeed, in one embodiment, the immobilization part and/or the core can be made of nearly pure aluminum such as Aluminum Association Type 1XXX aluminum, and preferably Type 1145 aluminum sheet material (99.45% pure) having a thickness in the range of 0.2 to 0.6 mm, and preferably having a thickness of about 0.4 mm. The aluminum metal can be annealed to a dead soft or “0” temper. Bending the aluminum core during the process of adjusting the flexible immobilization part 10 to conform to a patient's limb creates a curved cross-section that increases the rigidity of the splint, which makes it more resistant to bending.

In one embodiment of the invention, a padding material can be provided between the immobilization part 10 and the injured limb. The padding material is preferably a resiliently compressible or elastomeric material, and may be of a polymeric foam such as a closed cell microcellular low density expanded polyethylene available from Voltek Division of Sakisui American Corporation as its Volara Type A foam, with a layer of a flexible pressure-sensitive adhesive material already applied to one side of the foam to serve as the layer of adhesive. In one embodiment of the invention, the padding materials can be attached to or detached from the flexible immobilization part 10. For example, when attached, an adhesive material (e.g., a pressure-sensitive adhesive material, which may be acrylic based) can be used to secure the padding to the flexible part. The padding materials can extend past the length and width of the flexible part.

In one embodiment of the invention, the padding material can be made preferably of an open-cell polymeric foam, such as a polyurethane foam, with an applied layer of flexible pressure sensitive adhesive. The open-cell construction for the padding material allows circulation of air, to cool and to dissipate evaporation from the skin of a patient using the splint in order to provide comfort for the patient. One acceptable material is available from Foamex, of Compton, Calif., as its Foam Grade F 145 44 F.6 FA 44145-304.

In one embodiment of the invention, the padding materials can include multiple padding layers with the material contacting the limb being made of a soft, absorbent fabric with elasticity. For example, a brushed terrycloth or boucle fleece of 65 percent polyester and 35 percent rayon fiber of 100 denier yarn, available from Eclat Textile Co. Ltd. of City of Industry, Calif. as its product number 1206D is suitable.

In one embodiment of the invention, the immobilization part 10 is shaped to conform at least approximately to the patient's limb, prior to inflation of the air cuff. Since the core of the flexible immobilization part can be annealed to be dead soft after the initial manufacture of the splint, it is initially relatively easy to bend the immobilization part into the desired form. Accordingly, the form of the immobilization part 10 after shaping can have a curved cross-section that provides improved rigidity by acting structurally as a “C”-shaped channel. Once the shape of the immobilization part 10 has been properly adjusted, the immobilization part 10 is secured in place by wrapping the patient's limb with the splint structure 4 and fastening these components by way of the splint binding belts 6, as shown in FIG. 1. Inflation of the air cuffs will then immobilize the limb.

In some cases, it may be desired to provide additional stiffness in the portion of the flexible immobilization part by bending the immobilization part 10 outward to form side flanges. The degree of stiffness can be varied by folding.

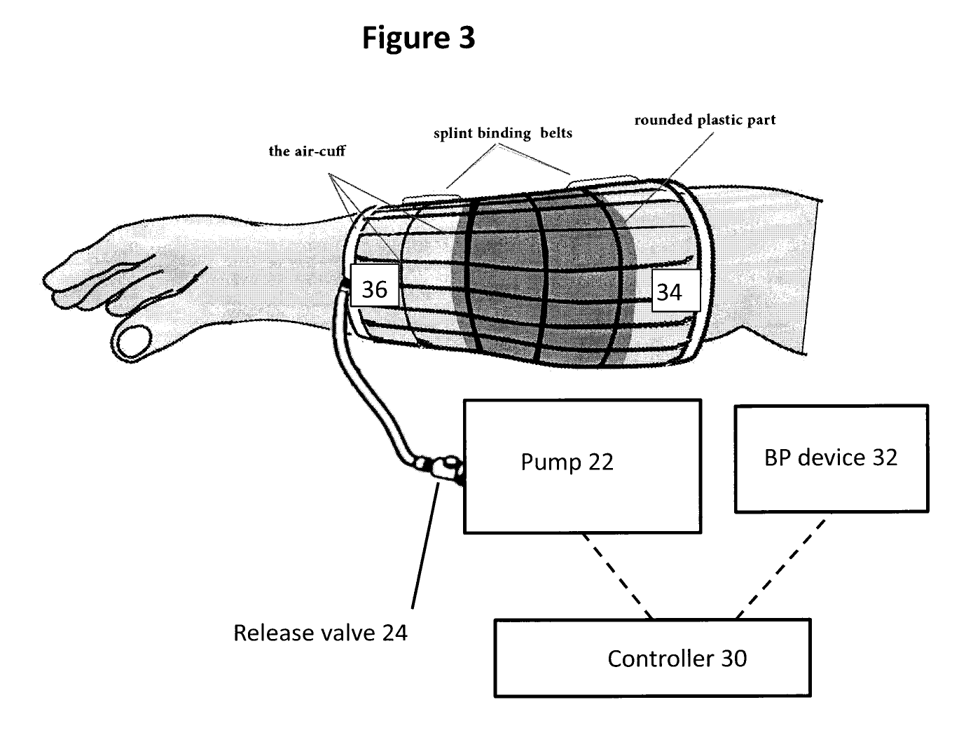

As shown in FIG. 3, in one embodiment of the invention, controller 30 is in communication with an automated blood pressure measurement device 32. The controller 30 can then determine a suitable air pressure that will not stop blood from flowing naturally in the veins of the area covered by the hollow expanded air cuff The automated blood pressure (BP) device 32 detects blood pressure readings from for example two sensors 34, 36. In one embodiment of the invention, sensor 34 is embedded in the front of the splint 2 (i.e., between the splint and the patient's heart). In one embodiment of the invention, sensor 36 is embedded in the rear of the splint 2 (i.e., on the opposite side of the splint from the patient's heart). Sensors 34, 36 (or other sensors included with the splint 2) send readings to controller 30 (e.g., a micro-chip processor as described in more detail below). In one embodiment of the invention, the sensors could be temperature sensors measuring the patient's temperature. In one embodiment of the invention, one or more sensors 34, 36 could be replaced with ultrasonic transducers to provide therapeutic energy to the injured limb.

In one embodiment of the invention, based on the sensor readings, controller 30 controls the air pressure applied so as not to cut off blood supply. However, in one embodiment of the invention, the controller can be set to cut off blood flow such that the splint 2 can act as a controllable tourniquet in emergency situations.

In one embodiment of the invention, there are body heat-absorbing electrical power generators 38, placed in the internal surface of the hollow expanded air cuff, to generate electricity from body heat of the patient. In one embodiment of the invention, the controllers and pumps can be powered in part or in total from the body heat-absorbing generators, or by connection to an external power source such as portable batteries. U.S. Pat. Appl. No. 20120211044 (the entire contents of which are incorporated by reference) describes thermoelectric devices attachable to the human body which are suitable here for this invention. In one embodiment of this invention, a thermoelectric conversion module (acting as a body heat-absorbing electrical power generator 38) is included in the outer or inner immobilization constructs (noted above) and placed in contact with the skin upon conforming the splint 2 in place around the limb. The thermoelectric conversion module could include: a first substrate having water permeability, a thermoelectric conversion element provided on the first substrate, a heat insulator having water permeability provided around the thermoelectric conversion element on the first substrate, and a second substrate disposed on the thermoelectric conversion element and the heat. The first or second substrate would contact the skin. The other side would then be exposed to air, thereby establishing a temperature difference across the thermoelectric conversion element.

When a temperature difference is present, a voltage potential difference occurs in each of the p-type semiconductor blocks and the n-type semiconductor blocks of the thermoelectric conversion module, and electric power can be extracted from module.

Features of the Exoskeleton Splint

This invention offers improvements into orthopedic splints. Current splints, particularly for limbs, are heavy and take time to install on the broken bone. In addition, current splints irritate the skin of the patient and prevent the patient from scratching the skin under the applied splints. Also, the smell of the splint has a tendency to get worse over time due to odor. Moreover, taking off splint requires tools that some time could be harmed to the patient and required skilled medical practitioner.

Additionally, once convention splints are in place, there is no capability to readjust the position of the securing force. Often, physicians have to wait for the swelling on a limb subsides before casting a broken or injured limb.

These disadvantages of the prior art are greatly eliminated by one or more of the embodiments and aspects of the invention discussed above.

Indeed, one feature of the exoskeleton splint is that it is breathable, while at the same time achieving proper immobilization of the limb. For example, adequate air circulation to the damaged area can occur by way of openings 16 permitting air to circulate over the skin in the area being immobilized. In one embodiment of this invention, splint 2 permits almost half of the traditional limb splint surface to be open so the skin can be exposed and, hence, reduce the amount of material and weight of the invention's splints.

Additionally, the time needed to install a splint on a limb is greatly reduced by this invention, making the splints of this invention especially attractive for emergency field use. Furthermore, the inflation process permits the splint to readily be re-positioned if needed.

Medical Kits and Method of Treating Injured Limbs

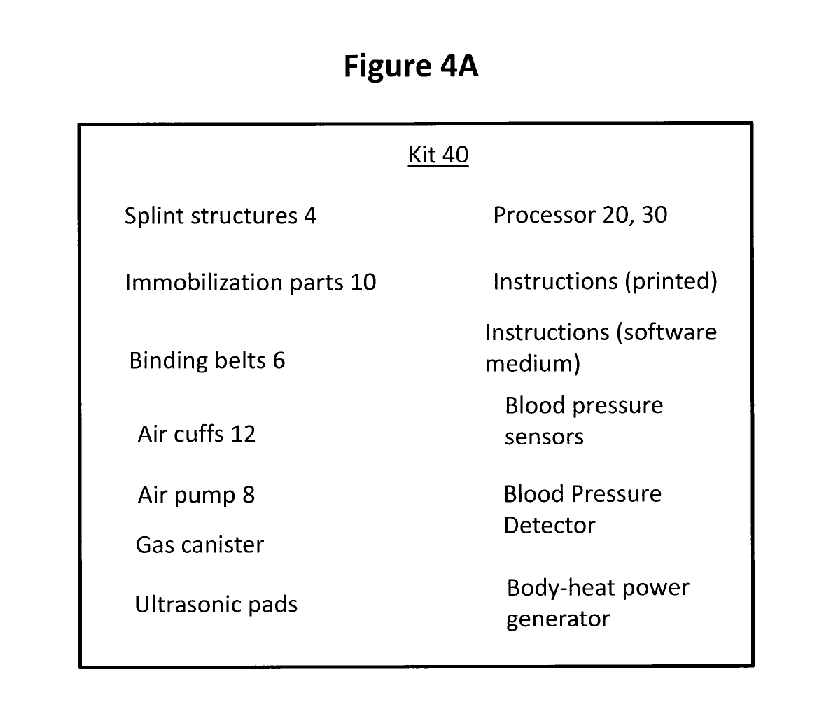

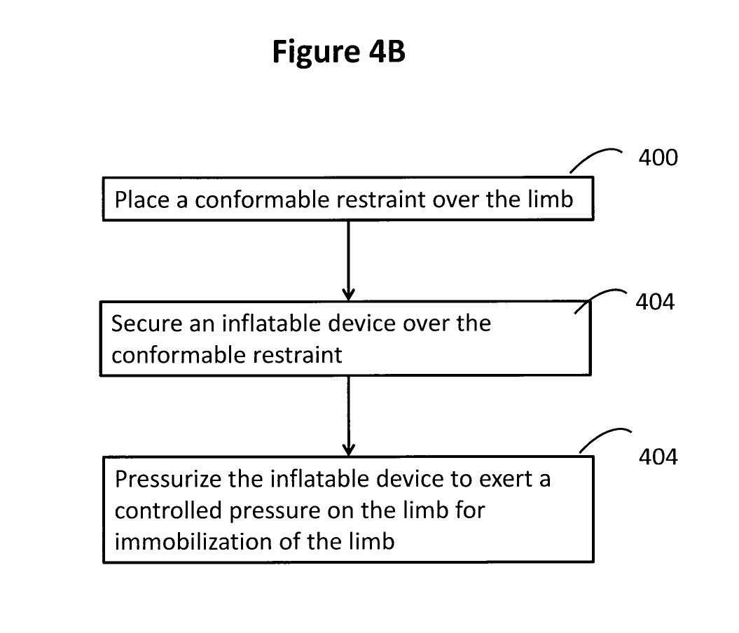

In one embodiment of the invention, as shown in FIG. 4A, there is provided a medical kit 40 containing those elements of the exoskeleton splint 2 noted above. More specifically, the kit includes a variety of sizes of the splint structures 4 and the immobilization parts 10 to accommodate fitting different size limbs and patients. Different binding belts 6 and air cuffs (if not integral to the splint structure 4 or the immobilization part 10 are included. Moreover, the kit can include the aforementioned pumps (manual or motorized), or the compressed gas container, for inflation of the air cuffs of the splint structures 4. Various sensors and other monitoring (such as thermometers) and therapeutic devices (such as ultrasonic transducers) can be included in the kit for attachment to the patient's skin secured for example by the air cuff or a clip to the inner or outer immobilization constructs (noted above).

In one embodiment of the invention, the kit can contain instructions for application and use of the exoskeleton splint 2. Accordingly, FIG. 4B is a flowchart depicting a medical procedure (i.e., method) for immobilization of a limb of a patient, which the kit's instructions would provide to a user. At step 400, a conformable restraint is placed over (beside) the limb. At step 402, an inflatable device is secured over the conformable restraint. At step 404, the inflatable device is pressurized to thereby exert a controlled pressure on the limb for immobilization of the limb.

In one embodiment of the invention, the kit's instructions could be in a software medium as described below. In this embodiment, the software medium would program the processors in controllers 20, 3 noted above to communicate 1) data on the patient such as blood pressure, pulse rate, body temperature or 2) data on the patient's pressure settings used to immobilize the limb to other remote devices or servers removed from the splint. Frequently, in today's world, patients in critical care wards have vital and treatment related data communicated wirelessly to central monitoring stations. Meanwhile, for in-the-field operations, communications from each installed limb to permit remote stations can help officials monitor the extent of injuries being treated in a given disaster area.

Computerized Control

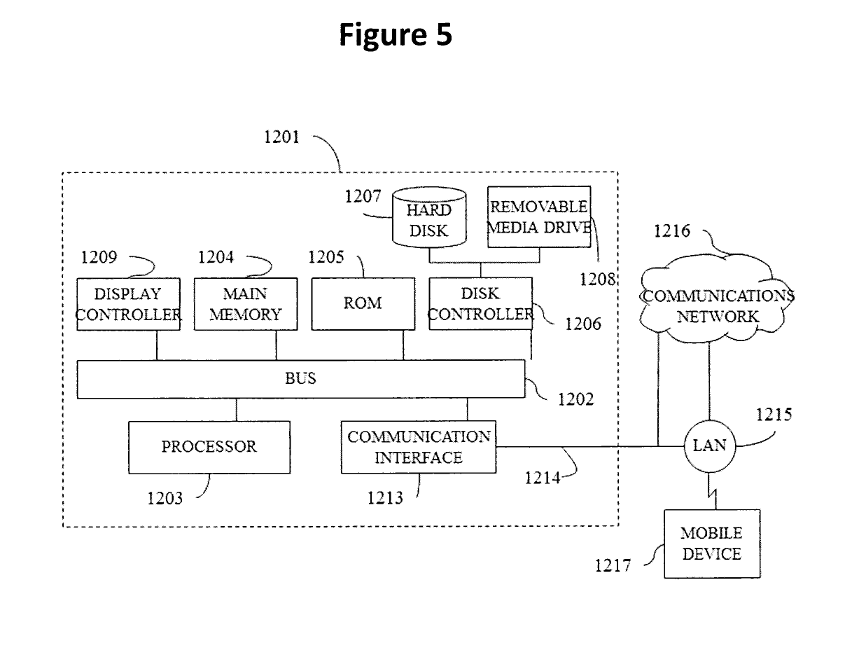

FIG. 5 is a schematic depicting a computer system 1201 for implementing various embodiments of the invention including that of operating the controllers 20 and/or 30, or for operation or communication with the blood pressure measurement device, the sensors, the ultrasonic transducers, the thermometers, the body-heat electric power generators, and the pump and release valve noted above.

The computer system 1201 includes a bus 1202 or other communication mechanism for communicating information, and a processor 1203 coupled with the bus 1202 for processing the information. The computer system 1201 also includes a main memory 1204, such as a random access memory (RAM) or other dynamic storage device (e.g., dynamic RAM (DRAM), static RAM (SRAM), and synchronous DRAM (SDRAM)), coupled to the bus 1202 for storing information and instructions to be executed by processor 1203. In addition, the main memory 1204 may be used for storing temporary variables or other intermediate information during the execution of instructions by the processor 1203. The computer system 1201 further includes a read only memory (ROM) 1205 or other static storage device (e.g., programmable read only memory (PROM), erasable PROM (EPROM), and electrically erasable PROM (EEPROM)) coupled to the bus 1202 for storing static information and instructions for the processor 1203.

The computer system 1201 also includes a disk controller 1206 coupled to the bus 1202 to control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display, such as a cathode ray tube (CRT), for displaying information to a user of splint 2.

The computer system 1201 performs operations of 1) pressure control for the air cuff and 2) data communications to remote sites in response to the processor 1203 executing one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, or any other medium from which a computer can read.

Stored on any one or on a combination of computer readable media, the invention includes software for controlling the computer system 1201, for driving a device or devices for implementing the invention, and for enabling the computer system 1201 to interact with a human user who for example may be monitoring a patient's status. Such software may include, but is not limited to, device drivers, operating systems, development tools, and applications software. Such computer readable media further includes the computer program product of the invention for performing all or a portion (if processing is distributed) of the processing performed in implementing the invention.

A computer readable medium may take many forms, including but not limited to, non-volatile media, volatile media, and transmission media. Non-volatile media includes, for example, optical, magnetic disks, and magneto-optical disks, such as the hard disk 1207 or the removable media drive 1208. Volatile media includes dynamic memory, such as the main memory 1204. Transmission media includes coaxial cables, copper wire and fiber optics, including the wires that make up the bus 1202. Transmission media also may also take the form of acoustic or light waves, such as those generated during radio wave and infrared data communications.

The computer system 1201 can also include a communication interface 1213 coupled to the bus 1202. The communication interface 1213 provides a two-way data communication coupling to a network link 1214 that is connected to, for example, a local area network (LAN) 1215, or to another communications network 1216 such as the Internet. These local and non-local communications permit the immobilization splint to be in communication with local devices in a rescue vehicle or a hospital room or to be in non-local communication to a central monitoring system which for example can use the body-heat generators and the blood pressure sensors to provide medical-personnel basic patient care information such as body temperature, pulse rate, and blood pressure. The communication interface 1213 may be a network interface card to attach to any packet switched LAN. As another example, the communication interface 1213 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. In one embodiment of this invention, this communication configuration provides the invention the capability to have multiple of the exoskeleton splints 2 networked together and in communication with each other and a health care provider. The local network 1214 and the communications network 1216 use, for example, electrical, electromagnetic, or optical signals that carry digital data streams, and the associated physical layer (e.g., CAT 5 cable, coaxial cable, optical fiber, etc). The signals through the various networks and the signals on the network link 1214 and through the communication interface 1213, which carry the digital data to and from the computer system 1201 may be implemented in baseband signals, or carrier wave based signals.

Generalized Statements of the Invention

The following numbered statements provide a general description of the invention and are not intended to limit the appended claims.

Statement 1. 1. A splint for immobilization of a limb of a patient, comprising:

an outer immobilization construct extending in a longitudinal direction of the limb;

an inner immobilization construct in contact with the limb;

a splint binding belt binding the outer immobilization construct in place around the second wrapping construct; and

an expandable air cuff disposed between the outer immobilization construct and the second immobilization construct, which upon inflation exerts pressure on the second immobilization construct to conform the inner immobilization construct to the limb.

Statement 2. The splint of statement 1, wherein the outer immobilization construct comprises a plurality of openings exposing the limb to air.

Statement 3. The splint of statement 2, wherein the air cuff comprises an interconnected network of inflatable passages having said openings interleaved in the network of inflatable passages.

Statement 4. The splint of statement 1, wherein the inner immobilization construct is inflexible perpendicular to the longitudinal direction of the limb and is flexible along the longitudinal direction of the limb so as to conform to the limb.

Statement 5. The splint of statement 4, wherein the inner immobilization construct comprises a molded plastic part.

Statement 6. The splint of statement 5, wherein molded plastic part comprises a cupped plastic part having a seam at inflection point which extends in the longitudinal direction.

Statement 7. The splint of statement 1, further comprising an air pump for inflation of the air cuff.

Statement 8. The splint of statement 7, wherein the air pump comprises at least one of a manual air pump or a motor driven air pump.

Statement 9. The splint of statement 7, wherein the motor driven air pump is driven by a battery.

Statement 10. The splint of statement 7, wherein the manual air pump comprises a squeezable bulb.

Statement 11. The splint of statement 7, wherein the manual air pump comprises a canister of compressed gas.

Statement 12. The splint of statement 1, further comprising a pressure release valve to release pressure from the air cuff if the pressure exceeds a predetermined threshold.

Statement 13. The splint of statement 1, further comprising a controller configured to control inflation of the air cuff.

Statement 14. The splint of statement 13, further comprising a pressure release valve controlled by said controller to release pressure from the air cuff if the pressure exceeds a predetermined threshold.

Statement 15. The splint of statement 14, further comprising an air pressure sensor configured to detect said predetermined threshold.

Statement 16. The splint of statement 14, further comprising at least one blood pressure sensor which detects a blood pressure of the patient.

Statement 17. The splint of statement 16, wherein the predetermined threshold is based on the detected blood pressure of the patient.

Statement 18. The splint of statement 16, wherein the at least one blood pressure sensor comprises an up-stream blood pressure sensor between a heart of the patient and the air cuff and a down-stream blood pressure sensor disposed on an opposite side of the air cuff from the heart.

Statement 19. The splint of statement 18, wherein the controller establishes said predetermined threshold based on values of blood pressure measured by the up-stream blood pressure sensor and the down-stream blood pressure sensor.

Statement 20. The splint of statement 1, further comprising body heat-absorbing generators which absorb heat and generate energy to power the splint.

Statement 21. A method for immobilizing a limb of a patient using one of the splints in statements 1-20, comprising:

placing a conformable restraint over the limb;

securing an inflatable device over the conformable restraint; and

pressurizing the inflatable device to thereby exert a controlled pressure on the limb for immobilization of the limb.

Statement 22. The method of statement 21, wherein pressurizing comprises inflating an air cuff to a pressure less than required to stop blood flow through the limb.

Statement 23. The method of Statement 21, wherein pressurizing comprises inflating an air cuff to a pressure sufficient to stop blood flow through the limb.

Statement 24. The method of statement 21, wherein pressurizing comprises progressively inflating an air cuff such that the conformable restraint conforms to the limb of the patent.

Statement 25. The method of statement 21, wherein pressurizing comprises progressively inflating an air cuff such that the conformable restraint exerts a force to set a bone in the limb.

Statement 26. The method of statement 21, wherein pressurizing comprises a first inflation of the inflatable device to evaluate immobilization of the limb, a deflation of the inflatable device, a repositioning of the conformable restraint, and a second inflation of the inflatable device.

Statement 27. The method of statement 21, wherein pressurizing comprises manually pumping air into the inflatable device or supplying air from a compressed gas source.

Statement 28. The method of statement 21, wherein pressurizing comprises pumping air from a motorized pump into the inflatable device.

Statement 29. The method of statement 21, further comprising determining a blood pressure in the limb of the patient and setting the controlled pressure to be lower than the blood pressure.

Statement 30. A kit containing at least one of 1) the splint of statements 1-20 and 2) instructions detailing the method for immobilization of statements 21-29.

Numerous modifications and variations of the present invention are possible in light of the above teachings. It is therefore to be understood that within the scope of the appended claims, the invention may be practiced otherwise than as specifically described herein.

1. A splint for immobilization of a limb of a patient, comprising:
an outer immobilization construct extending in a longitudinal direction of the limb;
an inner immobilization construct in contact with the limb;
a splint binder binding the outer immobilization construct in place around the second wrapping construct; and
an expandable air cuff disposed between the outer immobilization construct and the second immobilization construct, which upon inflation exerts pressure on the second immobilization construct to conform the inner immobilization construct to the limb.

2. The splint of claim 1, wherein the outer immobilization construct comprises a plurality of openings exposing the limb to air.

3. The splint of claim 2, wherein the air cuff comprises an interconnected network of inflatable passages having said openings interleaved in the network of inflatable passages.

4. The splint of claim 1, wherein the inner immobilization construct is inflexible perpendicular to the longitudinal direction of the limb and is flexible along the longitudinal direction of the limb so as to conform to the limb.

5. The splint of claim 4, wherein the inner immobilization construct comprises a molded plastic part.

6. The splint of claim 5, wherein molded plastic part comprises a cupped plastic part having a seam at inflection point which extends in the longitudinal direction.

7. The splint of claim 1, further comprising an air pump for inflation of the air cuff.

8. The splint of claim 7, wherein the air pump comprises at least one of a manual air pump or a motor driven air pump.

9. The splint of claim 7, wherein the motor driven air pump is driven by a battery.

10. The splint of claim 7, wherein the manual air pump comprises a squeezable bulb.

11. The splint of claim 7, wherein the manual air pump comprises a canister of compressed gas.

12. The splint of claim 1, further comprising a pressure release valve to release pressure from the air cuff if the pressure exceeds a predetermined threshold.

13. The splint of claim 1, further comprising a controller configured to control inflation of the air cuff.

14. The splint of claim 13, further comprising a pressure release valve controlled by said controller to release pressure from the air cuff if the pressure exceeds a predetermined threshold.

15. The splint of claim 14, further comprising an air pressure sensor configured to detect said predetermined threshold.

16. The splint of claim 14, further comprising at least one blood pressure sensor which detects a blood pressure of the patient.

17. The splint of claim 16, wherein the predetermined threshold is based on the detected blood pressure of the patient.

18. The splint of claim 16, wherein the at least one blood pressure sensor comprises an up-stream blood pressure sensor between a heart of the patient and the air cuff and a down-stream blood pressure sensor disposed on an opposite side of the air cuff from the heart.

19. The splint of claim 18, wherein the controller establishes said predetermined threshold based on values of blood pressure measured by the up-stream blood pressure sensor and the down-stream blood pressure sensor.

20. The splint of claim 1, further comprising body heat-absorbing generators which absorb heat and generate energy to power the splint.

21. A method for immobilizing a limb of a patient, comprising:
placing a conformable restraint over the limb;
securing an inflatable device over the conformable restraint; and
pressurizing the inflatable device to thereby exert a controlled pressure on the limb for immobilization of the limb.

22. The method of claim 21, wherein pressurizing comprises inflating an air cuff to a pressure less than required to stop blood flow through the limb.

23. The method of claim 21, wherein pressurizing comprises inflating an air cuff to a pressure sufficient to stop blood flow through the limb.

24. The method of claim 21, wherein pressurizing comprises progressively inflating an air cuff such that the conformable restraint conforms to the limb of the patent.

25. The method of claim 21, wherein pressurizing comprises progressively inflating an air cuff such that the conformable restraint exerts a force to set a bone in the limb.

26. The method of claim 21, wherein pressurizing comprises a first inflation of the inflatable device to evaluate immobilization of the limb, a deflation of the inflatable device, a repositioning of the conformable restraint, and a second inflation of the inflatable device.

27. The method of claim 21, wherein pressurizing comprises manually pumping air into the inflatable device or supplying air from a compressed gas source.

28. The method of claim 21, wherein pressurizing comprises pumping air from a motorized pump into the inflatable device.

29. The method of claim 21, further comprising determining a blood pressure in the limb of the patient and setting the controlled pressure to be lower than the blood pressure.

30. A kit containing the splint of claim 1 and instructions detailing a method for immobilization.
